(19) United States
(12) Patent Application Publication
(10) Pub. No.: US 2007/0141086 A1
(43) Pub. Date: Jun. 21, 2007

(54) Use of antibiotics as vaccine adjuvants

(76) Inventors: Michael K. Ohara, Kalamazoo, MI (US); David R. McGavin, Portage, MI (US); Randy D. Leyh, Richland, MI (US)

Correspondence Address: PHARMACIA & UPJOHN, 7000 Portage Road, KZO-300-104, KALAMAZOO, MI, 49001, US

(21) Appl. No.: 10/595,784

(22) PCT Filed: Nov. 8, 2004
(86) PCT No.: PCT/IB04/03694

§ 371 Date: May 11, 2006

Related U.S. Application Data
(60) Provisional application No. 60/524,097, filed on Nov. 21, 2003.

(51) Int. Cl. A61K 39/04; A61K 31/545; A61K 31/7048

(52) U.S. Cl. 424/248.1; 514/28; 514/200

(57) ABSTRACT

The invention describes an adjuvant composition comprising an antimicrobial agent, in particular an azalide, wherein the antimicrobial agent acts as an adjuvant. More particularly, the adjuvant composition is a vaccine adjuvant. The invention further describes a vaccine comprising several components comprising (a) at least one antigen and (b) at least one antimicrobial agent, in particular an azalide, wherein the azalide acts as an adjuvant. An adjuvant composition or vaccine of the present invention is useful in the prevention and treatment of diseases caused by a pathogenic agent, a cancerous cell, or an allergen.

### BACKGROUND OF THE INVENTION

The invention provides an adjuvant composition comprising at least one antimicrobial agent in particular an azalide, wherein the antimicrobial agent or azalide acts as an adjuvant. More particularly, the adjuvant composition is a vaccine adjuvant. The invention further provides a vaccine comprising (a) at least one antigen and (b) at least one antimicrobial agent, including an azalide, wherein the agent, acts as an adjuvant. An adjuvant composition or vaccine of the present invention is useful in the prevention and treatment of diseases caused by a pathogenic agents such as bacteria, e.g., M. haemolytica, protozoa, helminths, viruses, fungi, a cancerous cell or an allergen. The use of an azalide as an adjuvant has not yet been reported until Applicants' present invention.

### BRIEF DESCRIPTION OF THE DRAWINGS

FIG. 1. Geometric mean anti-leukotoxin antibody titer for each of the treatment groups.

FIG. 2. Least squares mean anti-whole cell antibody titer for each of the treatment groups.

### SUMMARY OF THE INVENTION

The invention provides an adjuvant composition comprising at least one antimicrobial or antibiotic agent, and especially an azalide, wherein the agent acts as an adjuvant. The agent may also provide therapeutic (e.g., antibiotic) properties; however, in a preferred embodiment of the invention, the agent provides little to no antimicrobial therapeutic properties. More particularly, the adjuvant composition is a vaccine adjuvant. The invention further provides a vaccine having two components comprising (a) at least one antigen and (b) at least one antimicrobial agent, wherein the antimicrobial agent acts as an adjuvant.

The antimicrobial agent for use in the present invention acts as an adjuvant, i.e., enhances, increases, upwardly modulates, diversifies or otherwise facilitates an immune response to an antigen. Numerous antimicrobial agents are suitable for this invention, including those listed herein. In one embodiment of the invention, the azalide is a 15-membered 9a-azalide having the following formula I: 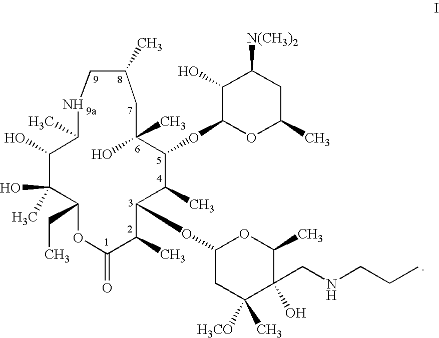
The chemical name of the compound of formula I is (2R,3S,4R,5R,8R,10R,11R,12S,13S,14R)-13-((2,6-dideoxy-3-C-methyl-3-O-methyl-4-C-((propylamino)-methyl)-α-L-ribo-hexopyranosyl)oxy-2-ethyl-3,4,10-trihydroxy-3,5,8,10,12,14-hexamethyl-11-((3,4,6-trideoxy-3-(dimethylamino)-β-D-xylo-hexopyranosyl)oxy)-1-oxa-6-azacyclopentadecan-15-one.

In another embodiment of the invention, the azalide is a mixture of azalides. Particularly, the azalide is a mixture of 9a-azalides. More particularly, the azalide is a mixture of 13- and 15-membered 9a-azalides. Even more particularly, the 9a-azalide mixture contains (a) a compound of formula I, as set forth above, and (b) a compound of formula II: 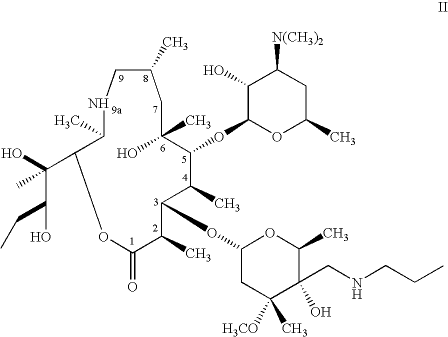
The chemical name of the 13-membered 9a-azalide of formula II is (3R,6R,8R,9R,10S,11S,12R)-11-((2,6-dideoxy-3-C-methyl-3-O-methyl-4-C-((propylamino)methyl-α-L-ribo-hexopyranosyl)oxy)-2-((1R,2R)-1,2-dihydroxy-1-methylbutyl)-8-hydroxy-3,6,8,10,12-pentamethyl-9-((3,4,6-trideoxy-3-(dimethylamino)-β-D-xylo-hexopyranosyl)oxy)-1-oxa-4-azacyclotridecan-13-one.

More particularly, the 9a-azalide mixture is a composition containing (a) a mixture of compounds of formulae I and II, each as set forth above, in a ratio of about, respectively, 90%±10% to about 10%±10%; preferably, 90%±4% to about 10%±4%; (b) water; and (c) one or more acids present at a total concentration of from about 0.2 mmol to about 1.0 mmol per mL of the composition. Such a composition may be prepared by heating to a temperature of about 50° C. to about 90° C. a mixture comprising: (i) the compound of formula (I), (ii) water, and (iii) one or more acids in a total amount ranging from about 0.2 mmol to about 1.0 mmol per mL of the mixture.

More particularly, the 9a-azalide mixture is a composition containing (a) (i) a mixture of compounds of formulae I and II, each as set forth above, in a ratio of about, respectively, 90%±10% to about 10%±10%; preferably, 90%±4% to about 10%±4%; (ii) water; and (iii) one or more acids present at a total concentration of from about 0.2 mmol to about 1.0 mmol per mL of the composition; and (b) one or more water-miscible co-solvents present in an amount of from about 250 to about 750 mg per mL of the composition. Such a composition may be prepared by heating to a temperature of about 50° C. to about 90° C. a mixture comprising the compound of formula I or II, each as set forth above, water and one or more acids in an amount ranging from about 0.2 mmol to about 1.0 mmol per mL of the mixture, wherein one or more water-miscible co-solvents is added before, during or after the heating step, in an amount of from about 250 to about 750 mg per mL of the composition. In a preferred embodiment, the water-miscible co-solvent is added after the heating step.

According to the invention, the concentration of the compound of formula I, in the 9a-azalide mixture composition set forth above, before the heating step ranges from about 50 mg per mL to about 500 mg per mL of the mixture. In a preferred embodiment thereof, the concentration ranges from about 50 mg/mL to about 200 mg/mL.

According to the invention, the concentration of the first mixture of compound I and compound II in the 9a-azalide mixture composition set forth above ranges from about 50 mg/mL to about 200 mg/mL of the composition. Particularly, the concentration of the first mixture of compound I and compound II in the 9a-azalide compositions set forth above ranges from about 75 to about 150 mg/mL, and more particularly from about 90 mg/mL to about 110 mg/mL of the composition.

The pH of the mixture ranges from about 5.0 to about 8.0, and more particularly, from about 5.0 to about 6.0. The heating takes place for about 0.5 to about 24 hours, and more particularly, from about 1 to about 8 hours.

Examples of suitable acids for the 9a-azalide mixture compositions set forth above include, but are not limited to, acetic acid, benzenesulfonic acid, citric acid, hydrobromic acid, hydrochloric acid, D- and L-lactic acid, methanesulfonic acid, phosphoric acid, succinic acid, sulfuric acid, D- and L-tartaric acid, p-toluenesulfonic acid, adipic acid, aspartic acid, camphorsulfonic acid, 1,2-ethanedisulfonic acid, laurylsulfuric acid, glucoheptonic acid, gluconic acid, 3-hydroxy-2-naphthoic acid, 1-hydroxy-2-naphthoic acid, 2-hydroxyethanesulfonic acid, malic acid, mucic acid, nitric acid, naphthalenesulfonic acid, palmitic acid, D-glucaric acid, stearic acid, maleic acid, malonic acid, fumaric acid, benzoic acid, cholic acid, ethanesulfonic acid, glucuronic acid, glutamic acid, hippuric acid, lactobionic acid, lysinic acid, mandelic acid, napadisylic acid, nicotinic acid, polygalacturonic acid, salicylic acid, sulfosalicylic acid, tryptophanic acid, and mixtures thereof. Particularly, the acid is citric acid. In a more particular embodiment, the citric acid is present in an amount of from about 0.02 mmol to about 0.3 mmol per mL of the composition. More particularly, the acid is a mixture of citric acid and hydrochloric acid. In a more particular embodiment, citric acid is present in an amount of from about 0.02 mmol to about 0.3 mmol per mL of the composition and the hydrochloric acid is present in an amount sufficient to achieve a composition pH of about 5 to about 6.

Examples of a suitable water-miscible co-solvent for the 9a-azalide mixture compositions set forth above include, but are not limited to, ethanol, isopropanol, diethylene glycol monomethyl ether, diethylene glycol butyl ether, diethylene glycol monoethyl ether, diethylene glycol dibutyl ether, polyethylene glycol-300, polyethylene glycol-400, propylene glycol, glycerine, 2-pyrrolidone, N-methyl 2-pyrrolidone, glycerol formal, dimethyl sulfoxide, dibutyl sebecate, polysorbate 80, and mixtures thereof. Particularly, the one or more water-miscible co-solvents is propylene glycol. More particularly, the propylene glycol is present in an amount of from about 450 to about 550 mg per mL of the composition.

In another particular embodiment, the one or more acids are citric acid present in an amount of from about 0.02 mmol to about 0.3 mmol per mL of the composition and hydrochloric acid is present in an amount sufficient to achieve a composition pH of about 5 to about 6; the one or more water-miscible co-solvents is propylene glycol present in an amount of from about 450 to about 550 mg per mL of the composition; and the azalide composition further comprises the antioxidant monothioglycerol present in an amount of from about 4 mg/mL to about 6 mg/mL of the composition.

Ceftiofur is another antibiotic that is particularly suited as an adjuvant. It is listed in Table 8, below and in other places herein. Ceftiofur is an antibiotic that is available in various salt forms and crystals; such as for example the sodium salt, hydrochloride form and a long acting version described as a crystal free acid form or CCFA. The long acting form is a particularly suitable form of the drug to act as an adjuvant because of its properties, including a long half life.

Each and every antibiotic listed in the tables herein, both individually and in combination with 1, 2, 3, 4 or 5 other antimicrobial agents are specifically described and claimed as a useful vaccine adjuvant or vaccine component herein.

According to the invention, the antigen can be any antigen which in combination with the antimicrobial, or in particular a macrolide, in particular an azalide or in particular a beta lactam and in particular, ceftiofur, elicits an enhanced, increased, upwardly modulated, diversified or otherwise facilitated immune response. Particularly the antigen stimulates the production of a specific antibody or antibodies that can combine with the antigen; and/or the antigen stimulates the generation of lymphocytes specific for the antigen, said lymphocytes then being able to react against the antigen by the production lymphokines that regulate and stimulate effector functions that can be targeted against the antigen or by the production of cells that can specifically react with the antigen. One ordinarily skilled in the art should be able to easily determine suitable antigens and numerous references are available to guide the practicioner, including the following: Clinical Microbiology and Infectious Diseases of the Dog and Cat, Greene, Craig E. 1984. W. B. Saunders Co. Diseases of Feedlot Cattle, Jensen, R., and Makay, Donald R. 1965. Lea and Febiger. Virus Infections of Carnivores, Appel, M. J. ed. 1987. Elsevier Science Publishers B. V. Virus Infections of Ruminants, Dinter, Z. and Morein, B. 1990. Elsevier Science Publishers B. V. Veterinary Virology (2nd edition) Fenner, F. J. et al., 1993. Academic Press, Inc. Infectious Diseases. A Treatise of Infectious Processes, Hoeprich, P. D. et al., 1994. J. B. Lippincott Co. Diseases of Swine, Leman, A. D. et al., 1992. Iowa State University Press. Diseases of Poultry, Calnek, B. W. (ed) 1997. Iowa State University Press. Feline and Canine Infectious Diseases, Gaskell, R. M. and Bennett, M. 1996. Blackwell Science Ltd. Diseases and Disorders of Cattle, Blowey, R. W. and Weaver, A. D. 1991. Wolfe Publishing Ltd.

Examples of suitable antigens are also defined herein. Particularly, the antigen may be M. haemolytica antigen, a M. haemolytica leukotoxin, a M. haemolytica capsular antigen, or a M. haemolytica soluble antigen, each as defined herein, or a mixture thereof (e.g., the One Shot® antigen, commercially available from Pfizer, Inc., New York).

The invention provides a method for enhancing, increasing, upwardly modulating, diversifying or otherwise facilitating an immune response to an antigen comprising administration of an adjuvant composition or vaccine adjuvant of the invention.

The invention provides a method for enhancing, increasing, upwardly modulating, diversifying or otherwise facilitating an immune response to an antigen comprising administration of a vaccine of the invention.

The invention further provides a method of treating disease caused by a pathogenic agent, a cancerous cell, or an allergen comprising the step of administering an adjuvant composition or vaccine adjuvant of the present invention.

The invention further provides a method of treating disease caused by a pathogenic agent, a cancerous cell, or an allergen comprising the step of administering a vaccine of the present invention.

The invention further provides a method of preventing disease caused by a pathogenic agent, a cancerous cell, or an allergen comprising the step of administering an adjuvant composition or vaccine adjuvant of the present invention.

The invention further provides a method of preventing disease caused by a pathogenic agent, a cancerous cell, or an allergen comprising the step of administering a vaccine of the present invention.

An adjuvant composition or vaccine adjuvant of the invention can be used in the manufacture of a medicament for the prophylactic treatment of a disease caused by a pathogenic agent, a cancerous cell, or an allergen.

An adjuvant composition or vaccine adjuvant of the invention can be used in the manufacture of a medicament for the therapeutic treatment of a disease caused by a pathogenic agent, a cancerous cell, or an allergen.

A vaccine of the invention can be used in the manufacture of a medicament for the prophylactic treatment of a disease caused by a pathogenic agent, a cancerous cell, or an allergen.

A vaccine of the invention can be used in the manufacture of a medicament for the therapeutic treatment of a disease caused by a pathogenic agent, a cancerous cell, or an allergen.

The invention here describes both human and non-human animal vaccines.

An adjuvant composition may comprising one or more antimicrobial agents. The human or non-human animal vaccine may comprise at least two components, with the two components administered either concurrently, or co-administered within a month, where the first component is an adjuvant comprising one or more antimicrobial agents and the second component is one or more antigenic agents.

A vaccine with adjuvant where the adjuvant is a antimicrobial agent which is a macrolide antibiotic. A vaccine where the vaccine is for non-human animals, where the antimicrobial agent is Draxxin® or tulthramycin, and where the antigenic agent is selected from one or more from the group consisting of a M. haemolytica antigen, a M. haemolytica leukotoxin, a M. haemolytica capsular antigen, a M. haemolytica soluble antigen, or a mixture thereof.

An adjuvant composition, that may be used in a vaccine, administered either concurrently or co-administered with an antigen selected from any M. haemolytica antigen with an adjuvant composition of claim 10, wherein said 9a-azalide is a composition comprising (a)(i) a mixture of compounds of formulae I and II in a ratio of about 90%±10% to about 10%±10%, respectively; (ii) water; and (iii) one or more acids present at a total concentration of from about 0.2 mmol to about 1.0 mmol per mL of the composition; and (b) one or more water-miscible co-solvents present in an amount of from about 250 to about 750 mg per mL of the composition.

A vaccine comprising any of the antimicrobial adjuvant compositions described here administered either concurrently or co-administered with an antigen.

A method for enhancing, increasing, upwardly modulating, diversifying or otherwise facilitating an immune response in an animal to an antigen comprising administration of an antimicrobial agent to an animal.

A vaccine where an antimicrobial agent is at least one adjuvant component of a concurrent, or co-administration of an antimicrobial agents and an antigen, where the antimicrobial agent is selected from the antimicrobial agents described herein, and where the antigenic agents are described herein.

A method of preventing a disease caused by a pathogenic agent, cancerous cell, or allergen in an animal comprising the step of administering the adjuvant compositions or vaccines described herein to an animal susceptible to said disease. The preparation of a medicament of the type described herein to create a vaccine or kit. The use of such a preparation of vaccine or kit to vaccinate an animal against disease.

A kit comprising the adjuvant or vaccines described herein, where the components of the kit has either an antimicrobial agent or an antigenic agent or both and where said components that can be either co-administered or concurrently administered, with instructions for use thereof.

### DETAILED DESCRIPTION OF THE INVENTION

### Definitions

As used herein, the article “a” or “an” refers to both the singular and plural form of the object to which it refers.

As used herein, the term “adjuvant”, unless indicated otherwise, refers to any substance or mixture of substances that enhances, increases, upwardly modulates, diversifies or otherwise facilitates the immune response (e.g., humoral or cellular immune response) to an antigen.

The term “antigen” or “antigenic agent”, unless indicated otherwise, refers to any agent that, when introduced into an immunocompetent human or animal, stimulates a humoral and/or cell mediated immune response. The antigen may be a pure substance, a mixture of substances, or particulate material (including cells, cell fragments, or cell derived fragments) or a live, usually attenuated, organism or virus. Examples of suitable antigens include, but are not limited to, a protein, glycoprotein, lipoprotein, peptide, carbohydrate/polysaccharide, lipopolysaccharide, toxin, virus, bacterium, fungus, and parasite. Other suitable antigens include minimal components of an antigen such as, but not limited to, an antigenic determinant, epitope, or peptide. Still other suitable antigens include those described in U.S. Pat. No. 5,855,894. An antigen may be native (naturally expressed or made), synthetic or derived by recombinant DNA methodologies familiar to those skilled in the art.

The term “antimicrobial agent” refers to any agent that kills or suppresses the multiplication or growth of a microorganism—which includes bacteria, e.g., M. haemolytica, protozoa, helminths, viruses, fungi, a cancerous cell or an allergen. It is a chemical substance that is sufficiently non-toxic to the host as to be useful for internal or external administration. Examples of antimicrobial agents are provided and named in detail below, but the invention also includes any such agent either described here or later discovered. One particular type of antimicrobial agent is an antibiotic especially useful as an adjuvant, those are azalides. Another preferred antimicrobial agent are beta lactams, in particular ceftiofur, and more particular the long acting ceftiofur. The time period of administration or duration of the antimicrobial agent is related to its potency and the period of administration for antimicrobial use. Typically it will be administered 1 to 3 times a day for about a week plus or minus a few days. In a preferred embodiment only one administration antibiotic adjuvant is needed. In a more preferred embodiment the one administration may be given at the about the same time as the vaccine component, either in the same syringe or applicator or in a separate syringe or applicator administered at about the same time as the other component or vaccine. In various embodiments the time period may be anywhere from about the same time, about 1 to 2 hours or 1 to 10 days with specific periods of within about 1, 2, 3, 4, 5, 6, 7, 8 hours or about 1, 2, 3, 4, 5, 6, 7, 8, 9, or 10 days being particularly and individually described and claimed herein. One ordinarily skilled in the art should be able to easily determine the length of time of administration of the antimicrobial agent.

The term “azalide”, unless indicated otherwise, refers to the class of compounds characterized by sugar(s)-substituted nitrogen-containing macrocyclic lactone rings. Examples of suitable azalides include, but are not limited to, 8a- and 9a-azalides and mixtures thereof. Particularly, the azalide is an 8a-azalide, a 9a-azalide or a mixture thereof. Examples of suitable 8a-azalides include, but are not limited to, those described in U.S. Pat. No. 6,054,434. Examples of suitable 9a-azalides include, but are not limited to, those described in U.S. Pat. Nos. 6,339,063 and 6,514,945.

The term “capsular antigen”, unless indicated otherwise, refers to any of the antigens, usually polysaccharide in nature, that are carried on the surface of bacterial capsules. Capsular antigen may alternatively referred to as a capsular polysaccharide or capsular substance. For example, a capsular antigen can be a soluble capsular polysaccharide from M. (P.) haemolytica as described in the literature. See e.g. Inzana, T. J., “Capsules and Virulence in the HAP Group of Bacteria” Can J of Vet Research, 54:S22-S27 (1990); and Adlam et al., “Purification, characterization and immunological properties of the serotype-specific capsular polysaccharide of Pasteurella haemolytica (serotype A1) organisms” J Gen Microbiol, 130:2415-2426 (1984).

The term “ceftiofur” refers to an antimicrobial antibiotic of the cephalosporin types. All cephalosporins are claimed and described here. The concentration of the cephalosporin in the formulation of the present invention may vary between about 1 mg/ml to 500 mg/ml. Preferably, for example, for ceftiofur hydrochloride, the concentration is about 50 mg/ml. In general, the upper limit on the concentration is determined by when the oil composition becomes too viscous to syringe. Additional information on the dosage and mode of administration of the antibiotic ceftiofur hydrochloride is contained in U.S. Pat. No. 4,902,683, which is hereby incorporated by reference herein.

Ceftiofur hydrochloride formulations at a concentration of 12.5 mg/ml are also available. Where the antibiotic is ceftiofur or a pharmaceutically acceptable salt thereof, a preferred concentration range in a composition of the invention is about 1 to about 1000 mg/ml, more preferably about 5 to about 750 mg/ml, and still more preferably about 10 to about 100 mg/ml. For antibacterials other than ceftiofur, suitable concentration ranges that are antibacterially equivalent can be determined by one of skill in the art based upon published data.

Ceftiofur is a powerful antibiotic available in several forms, sodium salt, HCl and free acid and polyforms, all salts and forms are claimed here. For the purpose of this invention the most preferred for is the crystalline free acid (CCFA). The desired level of ceftiofur metabolites in the patient's blood plasma is noted to be maintained at or above about 0.2 μg/ml. In one embodiment of the invention, a single dose of sustaining-vehicle CCFA maintains a ceftiofur metabolite level in the blood plasma of at or above about 0.2 μg/ml for at least three and preferably at least about four and more preferably at least about five days post-administration (sustained delivery of CCFA). Comparisons as to the degree of sustained delivery are made with equivalent bioactive agents. That is, sodium salts to sodium salts and free bases to free bases. Sustained-delivery should be be specifically reconciled with the regulatory definition for the same term that requires that the concentration versus time profile have three distinct phases (i.e., an increasing concentration phase, a plateau phase and a concentration depletion phase). While the term sustained-delivery may encompass the above regulatory definition it is not intended to be limited to it as compositions which are sustained delivery as defined herein need not possess the three distinct phases (e.g., the composition may have an increasing concentration phase and an extended concentration depletion phase). The amount of inventive composition to be administered is that which will deliver the bioactive agent in an amount and for a duration to provide a therapeutic benefit necessary to treat or prevent a disease without causing toxicity problems to the patient. The specific amounts to be selected are deemed to be within the skill of the artisan. For example, when CCFA is selected as the bioactive agent, it is administered in unit dosage form for intramuscular or subcutaneous administration comprising about 0.5 to about 10.0 mg CCFA/kg body weight of patient with preferred ranges of about 4.4-6.6 mg/kg for cattle, and 5.0-7.5 mg/kg for swine. To the extent necessary for completion the dosages as described in U.S. Pat. No. 5,721,359 and U.S. Pat. No. 6,074,657 are expressly incorporated by reference.

The term “concurrent administration” unless indicated otherwise, refers to the administration of one component of this invention, such as the adjuvant, within a certain time period of the other component, such as the vaccine. The site of administration of the two components on the animal can be any suitable site or route of administration. Typically the time period is 10 days or less, more preferable a week plus or minus a few days, more preferable 2, 3, 4, 5, 6. In various embodiments the time period may be anywhere from about 2 to 10 days with specific periods of about 2, 3, 4, 5, 6, 7, 8, 9, or 10 days being particularly described. The components may be administered in one, two, or more syringes.

The term “co-administration” unless indicated otherwise, refers to the administration of one component of this invention, such as the adjuvant, within a certain time period of the other component, such as the vaccine. The site of administration of the two components on the animal can be any suitable site or route of administration. Typically the time period of the two components may be at about the same time, or within an hour. In various embodiments the time period may be anywhere from about 0, 1, or 2 hours, preferred, but also specifically described and claimed are about 1, 2, 3, 4, 5, 6, 7, or 8 hours in the same day, with each possible time period being particularly and individually described and claimed herein. The time period may be up to 1 day for a co-administration of the two components. The components may be administered in one, two, or more syringes or applicators. More preferably the components may be administered with one or two syringes within an hour. More preferable at about the same time. The two components may be in the same or different syringes.

The term “kit” refers to any set or collection of articles for a specific purpose, here to immunize a human or animal. It can include and refer to a container for such a kit. It may refer to a packaged set of materials including vials and instructions or directions. It may be in one or more parts with the parts of the kit divided into discrete areas of the package. There may be one or more packages that contain or make up any particular kit.

The term “leukotoxin”, unless indicated otherwise, refers to any compound toxic to leukocytes. For example, the leukotoxin can be a soluble toxin produced by actively growing Mannheimia (Pasteurella) haemolytica as taught in the literature. See e.g., U.S. Pat. No. 5,055,400; Canadian patent application 91000097 and Gentry et al., “Neutralizing monoclonal antibodies to P. haemolytica leukotoxin affinity-purify the toxin from crude culture supernatants” Microbial Pathogenesis, 10: 411-417 (1991). “Leukotoxoid” is the term used to describe inactivated leukotoxin. Leukotoxin is alternately referred to in the literature by other identifiers as exotoxin or cytotoxin.

The term “soluble antigen”, unless indicated otherwise, refers to any antigen(s) from any source that exists or can exist in a soluble state. For example, a soluble antigen can be a soluble antigen shed during growth of M. (P.) haemolytica other than leukotoxin and capsular antigen such as glycoprotease and neuramindase. See e.g. Reggie et al. “Molecular Studies of Ssal, a Serotype-Specific Antigen of Pasteurella haemolytica A1”, Infection and Immunity, Vol. 59 No. 10 3398-3406 (1991).

The term “tulathromycin”, unless indicated otherwise, refers to 9a-azalide mixture composition containing (a)(i) a mixture of compounds of formulae I and II, each as set forth above, in a ratio of about 90%±4% to about 10%±4%, respectively; (ii) water; and (iii) one or more acids present at a total concentration of from about 0.2 mmol to about 1.0 mmol per mL of the composition; and (b) one or more water-miscible co-solvents present in an amount of from about 250 to about 750 mg per mL of the composition.

The term “vaccine”, unless indicated otherwise, refers to any preparation of antigen or immunogenic material suitable for the stimulation of active immunity in animals or humans. An antimicrobial agent or composition of vaccine adjuvant and in particular an azalide composition or vaccine adjuvant of the present invention may be used in such a preparation.

The antimicrobial agent or composition of vaccine adjuvant and in particular an azalide for use in the present invention, as set forth above, may be commercially available or prepared by using organic chemical reactions and techniques known in the art, including the methods described above. For example, the azalide of formula I, as set forth above, can be formed from a translactonization reaction of the azalide of formula II, as set forth above. Likewise, the azalide of formula II can be formed from a translactonization reaction of the azalide of formula I. Mixtures of the azalide of formulae I and II can be obtained from either a compound of formula I or formula II upon equilibration in an aqueous solution. Methods for obtaining the azalide of formula I are described in International publication no. WO 98/56802. Methods for obtaining the azalide of formula II are described in U.S. Pat. No. 6,514,945. Other methods for preparing azalides are described in U.S. Pat. Nos. 6,054,434 and 6,339,063 as well as the methods described in the examples set forth below.

A vaccine of the present invention may be prepared by any means known in the art including the procedure set forth in Example 1 below. Particularly, a vaccine may be prepared by combining at least one azalide with at least one antigen, each as set forth herein. More particularly, the antigen is in freeze-dried form and is reconstituted with at least one azalide solution acting as an adjuvant just prior to use. Alternatively, a solid (e.g., powder) azalide (e.g., a compound of either formula I or II) is combined with an aqueous antigen solution to form the vaccine.

An adjuvant composition, vaccine adjuvant or vaccine of the present invention may further contain additional agents. For example, additional antigens may be present. For example, an adjuvant composition, vaccine adjuvant or vaccine of the present invention may contain a combination of antigens from Pasteurella multocida, Haemophilus somni, Clostridial species, Mycoplasma species, Bovine Respiratory Syncytial Virus, Bovine Viral Diarrhea Virus, and/or Bovine Parainfluenza Type 3 virus, or any other infectious agent or derivative thereof. An adjuvant composition, vaccine adjuvant or vaccine of the present invention can also contain antigen(s) related to, derived from, or identical to, an antigen from a cancer cell or an allergen.

The adjuvant composition, vaccine adjuvant or vaccine of the present invention may further comprise one or more antioxidants present in an amount of from about 0.01 mg to about 10 mg per mL of the composition. Particularly, the one or more antioxidants is selected from the group consisting of sodium bisulfite, sodium sulfite, sodium metabisulfite, sodium thiosulfate, sodium formaldehyde sulfoxylate, L-ascorbic acid, erythorbic acid, acetylcysteine, cysteine, monothioglycerol, thioglycollic acid, thiolactic acid, thiourea, dithiothreitol, dithioerythreitol, glutathione, ascorbyl palmitate, butylated hydroxyanisole, butylated hydroxytoluene, nordihydroguaiaretic acid, propyl gallate, alpha-tocopherol, and mixtures thereof. More particularly, the one or more antioxidants is monothioglycerol. In another particular embodiment, monothioglycerol is present in an amount of from about 4 mg/mL to about 6 mg/mL of the composition.

The adjuvant composition, vaccine adjuvant or vaccine of the present invention may further comprise one or more preservatives in an amount of from about 0.01 to about 10 mg per mL of the composition. Examples of suitable preservatives include, but are not limited to, benzalkonium chloride, benzethonium chloride, benzoic acid, benzyl alcohol, methylparaben, ethylparaben, propylparaben, butylparaben, sodium benzoate, phenol, and mixtures thereof. As would be understood by one of skill in the art, the presence or absence of a preservative will depend upon the antigen. For example, if the antigen is a live bacterial antigen, then no preservative would be added.

The adjuvant composition, vaccine adjuvant or vaccine of the present invention may further comprise an additional non-antimicrobial agent adjuvant and in particular non antimicrobial and non-azalide adjuvant. Examples of suitable non-microbial and non-azalide adjuvants include those known in the art.

An adjuvant composition of the invention may be administered as part of a vaccine formulation, which may optionally contain an additional adjuvant. Alternatively, an adjuvant composition of the invention may be administered in addition to, i.e., separately, a vaccine, which may optionally contain an “additional adjuvant” being an adjuvant other than the adjuvant composition of the invention. Regardless of mode of administration, the antimicrobial agent and in particular the azalide acts as an adjuvant or provides an adjuvant effect, i.e., elicits an enhanced, increased, upwardly modulated, diversified or otherwise facilitated immune response to an antigen.

The adjuvant composition, vaccine adjuvant or vaccine of the present invention may be used to prevent or treat diseases in humans or animals caused by a pathogenic agent, a cancerous cell, or an allergen by the administration of a therapeutically effective amount of the adjuvant composition or vaccine to the human or animal susceptible to the disease.

According to the invention, the pathogenic agent may be any pathogenic agent including, but not limited to, bacteria, protozoa, helminths, viruses and fungi. Diseases in animals caused by such pathogenic agents include, but are not limited to, bovine respiratory disease, swine respiratory disease, pneumonia, pasteurellosis, coccidiosis, anaplasmosis, and infectious keratinitis. Thus, the adjuvant compositions and vaccine adjuvants of the invention can be used to prevent or treat, inter alia, bovine respiratory disease, swine respiratory disease, pneumonia, pasteurellosis, coccidiosis, anaplasmosis, and infectious keratinitis.

According to the invention, the cancerous cell may be any type of cancerous cell in the art. According to the invention, the allergen may be any allergen known in the art.

The adjuvant composition, vaccine adjuvant or vaccine of the invention can be used to protect or treat human and non-human animals such as both livestock animals and domestic animals including, but not limited to, cattle, horses, sheep, swine, goats, rabbits, cats, dogs, and other mammals in need of treatment. The adjuvant composition, vaccine adjuvant or vaccine of the invention can be also used to protect or treat humans. As would be understood by one of skill in the art, the adjuvant composition and/or vaccine of the invention to be administered will be chosen based on the patient to be protected or treated. Thus, as would be understood by one of skill in the art, an adjuvant composition, vaccine adjuvant or vaccine of the invention used for the protection or treatment of animals may differ from the adjuvant composition, vaccine adjuvant or vaccine of the invention used for the protection or treatment of humans.

The adjuvant composition, vaccine adjuvant or vaccine may be administered through oral, intramuscular, intravenous, subcutaneous, intra-ocular, parenteral, topical, intravaginal, or rectal routes. For administration to cattle, swine or other domestic animals, the adjuvant compositions or vaccine adjuvants may be administered in feed or orally as a drench composition. Particularly, the adjuvant composition, vaccine adjuvant or vaccine is injected intramuscularly, intravenously or subcutaneously.

For purposes of this invention, a therapeutically effective amount is that amount which enhances, increases, upwardly modulates, diversifies or otherwise facilitates an immune response to an antigen. Particularly, a therapeutically effective amount is that amount which induces immunity in the animal susceptible to the disease caused by the pathogenic agent, cancerous cell, or allergen. As would be understood by one of skill in the art, a therapeutically effective amount will vary and be determined on a case-by-case basis. Factors to be considered are the same as those outlined below for determining proper dosages. For example, a therapeutically effective amount can be readily determined by testing a variety of adjuvant compositions or vaccine preparations made in accordance with this invention in cattle and selecting the composition or vaccine preparation that induced immunity in a statistically significant number of cattle when challenged with M. (P.) haemolytica. A vaccine induced immunity can be measured by resistance to experimental challenge reflected by decreased or absence of mortality, absence of, or minimal clinical signs, reduction or complete elimination of characteristic lung lesions as is known to those in the art.

Typically the dosages and amounts of antimicrobial agents to be used can be determined by one ordinarily skilled in the art. More potent and longer lasting antimicrobials would not need as great an amount as antibiotics that have shorter half-lives or are less potent. To guide the user here we provide included Tables with typical dosages and interval times. The amount of such adjuvant to be used and its frequency of administration is also described elsewhere in this document. By way of example only and without limiting this invention specific recommended dosages for one type of antimicrobial, azalides are provided here.

Particularly, the azalide adjuvant composition or vaccine adjuvant, whether co-administered or concurrently administered, and in particular Draxxin® may be administered in dosages ranging from about 0.01 mg of the equilibrium mixture of compounds per kg of body weight (mg/kg) to about 20 mg/kg. More particularly, the adjuvant composition or vaccine adjuvant, whether co-administered or concurrently administered, may be administered in dosages ranging from about 1 mg/kg to about 10 mg/kg. Even more particularly, the adjuvant composition or vaccine adjuvant, whether co-administered or concurrently administered, are administered in dosages ranging from about 1.25 mg/kg to about 5.0 mg/kg.

Ceftiofur is another antibiotic that is particularly suitable for the purposes described in this document. It is listed in Table 8, below and in other places herein, especially and specifically described in the “Definitions” section under “Ceftiofur.” Ceftiofur is an antibiotic that is available in various salt forms and crystals; such as for example the sodium salt, hydrochloride form and a long acting version described as a crystal free acid form or CCFA. The long acting form is a particularly suitable form of the drug to act as an adjuvant because of its properties, including a long half life.

Several names and formula for ceftiofur are provided. The structure of ceftiofur hydrochloride is as follows:

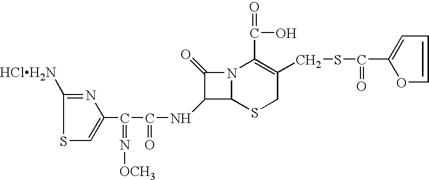

This compound is a crystalline hydrochloride salt of 7-[2-(2-amino-1,3-thiazol-4-yl)-2-methoxyimino)acetamido]-3-[(fur-2-ylcarbonyl)thiomethyl]-3-cephem-4-carboxylic acid. This cephalosporin free acid compound is known by the generic name, ceftiofur. Its preparation is described in U.S. Pat. No. 4,902,683, Amin et al., 20 Feb. 1990, which is hereby incorporated by reference.

The structure of ceftiofur free acid is Formula II as follows:

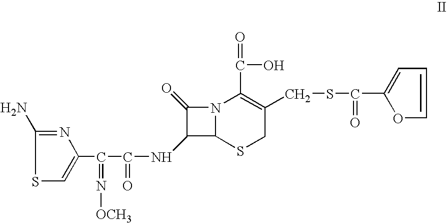

This compound is a crystalline free acid form of ceftiofur. Its preparation is described in International Publication No. WO 94/20505, published 15 Sep. 1994, Dunn et al., which is hereby incorporated by reference.

The adjuvant composition or vaccine adjuvant, whether co-administered or concurrently administered, may be administered continuously, intermittently or as a single dose. Those of skill in the art will readily recognize that variations in dosages and length of treatment can occur depending upon the species, weight and condition of the subject being treated, its individual response to the adjuvant compositions and vaccines, and the particular route of administration chosen. In some instances, dosage levels below the lower limit of the aforesaid ranges may be therapeutically effective, while in other cases still larger doses may be employed without causing any harmful side effects, provided that such larger doses are first divided into several small doses for administration throughout the day. A booster dose is believed desirable whenever subsequent stress or exposure is likely. The mode of administration of the adjuvant compositions or vaccine adjuvants, whether co-administered or concurrently administered, may be any suitable route which delivers the adjuvant compositions, whether co-administered or concurrently administered, to the host. Subcutaneous administration or administration by intramuscular injection is preferred.

The following Examples further illustrate the compositions and methods of the present invention. It is to be understood that the present invention is not limited to the specific details of the Examples provided below.

### EXAMPLE 1

### Vaccine Preparations and Treatments

An expired, commercial Mannheimia haemolytica vaccine (One Shot®, commercially available from Pfizer, Inc., New York) antigen was used as a model antigen in these studies. This antigen was reconstituted using One Shot® adjuvant, sterile water or tulathromycin. Saline was used as a negative control. Vaccine efficacy was evaluated by serology and by challenge with a virulent isolate of M. haemolytica. Forty beef calves weighing an average of 478 pounds on Day-1 were enrolled in the study. Calves were selected based on having low antibody titers to leukotoxin. Treatment study groups are shown in Table 1.

TABLE 1
Vaccine and treatment groups.

| Treat- | | | | Number of |
|---|---|---|---|---|
| ment | | Dose | | animals |
| Group | Vaccine | Volume | Route | vaccinated |
| T01 | Saline | 2 ml | SC | 10 |
| T02 | One Shot ® vaccine | 2 ml | SC | 10 |
| T03 | One Shot ® antigen reconstituted | 2 ml | SC | 10 |
| | in sterile water | | | |
| T04 | One Shot ® antigen reconstituted | 5.3 ml | SC | 10 |
| | in tulathromycin | | | |

This study was designed to evaluate the adjuvant properties of the 9a-azalide tulathromycin by replacing the adjuvant in a commercial Mannheimia haemolytica vaccine (One Shot® with tulathromycin.

Each animal was injected subcutaneously on the left side of the neck on Day 0. A 2-ml dose of saline solution was administered to each animal in T01. One Shot® Mannheimia (Pasteurella) haemolytica Bacterin-Toxoid was reconstituted in One Shot® adjuvant and administered to the T02 calves. The vaccine was reconstituted using sterile water and administered to T03 animals. One Shot® Mannheimia (Pasteurella) haemolytica Bacterin-Toxoid was reconstituted in tulathromycin. The mean body weight of the calves in T04 on Day-1 was 471.1 pounds. A volume of 5.3 ml of tulathromycin was used per dose to reconstitute the vaccine and was administered to each calf in T04.

For these studies animals were allocated to treatments per a randomized complete block design. The blocking factor was based upon leukotoxin serology titers obtained prior to the start of the study. Serology data was summarized by time-point. A log transformation {ln(n+1)} was applied to titer values prior to analysis. Linear combinations of the parameter estimates were used in a priori contrasts after testing for either a significant (P≦0.05) treatment effect or interaction effect between time-point and treatment. Comparisons were made between treatments at each time-point. The 5% level of significance (P≦0.05) was used to assess statistical differences. 95% confidence intervals for each of the mean values were also calculated. For titer values, geometric means at each sampling time-point were calculated from least squares means of the ln(titer values+1).

### EXAMPLE 2

### Post-Vaccination Serology

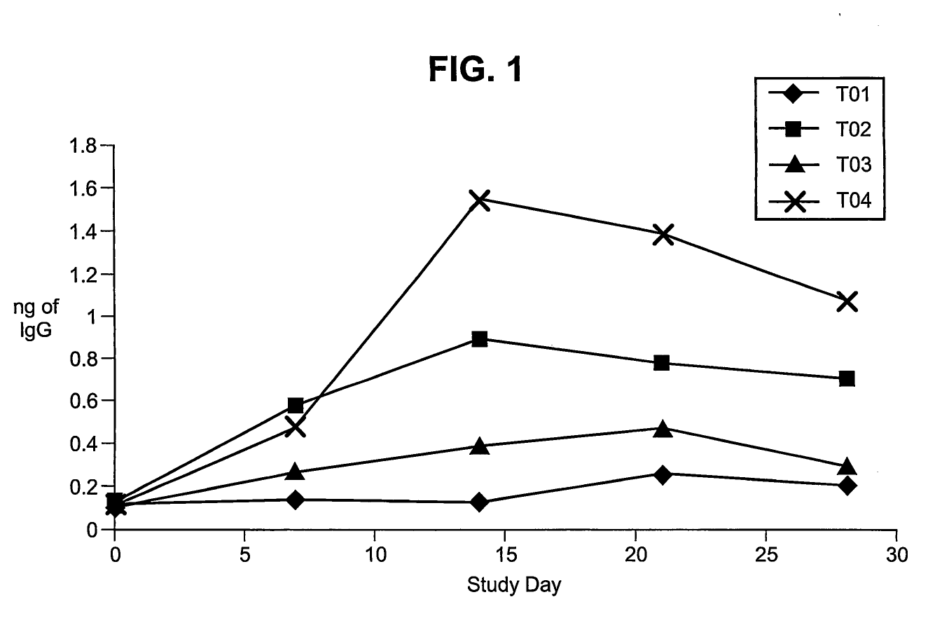

Serum anti-leukotoxin antibodies were monitored after vaccination (See Table 2; FIG. 1). Following vaccination the anti-leukotoxin mean antibody level in ng of IgG (See Confer, et al., “Serum antibody responses of cattle to iron-regulated outer membrane proteins of Pasteurella haemolytica A1” Vet Immunol Inmunopathol Vol. 47, pp 101-110 (1995)) significantly increased by Day 7 in both T02 and T04 compared to the controls and remained higher throughout the study (P≦0.05). On Days 14 and 21, the T04 mean anti-leukotoxin antibodies were significantly higher than those in T02 (P≦0.05). Although the mean antibody levels remained higher in T04 compared to T02 for the rest of the study, the difference between the two groups decreased. The level of anti-leukotoxin antibodies in T01 was relatively unchanged during the study. The antibody levels in T03 were not significantly different from the T01 levels on any of the sample days (P>0.05).

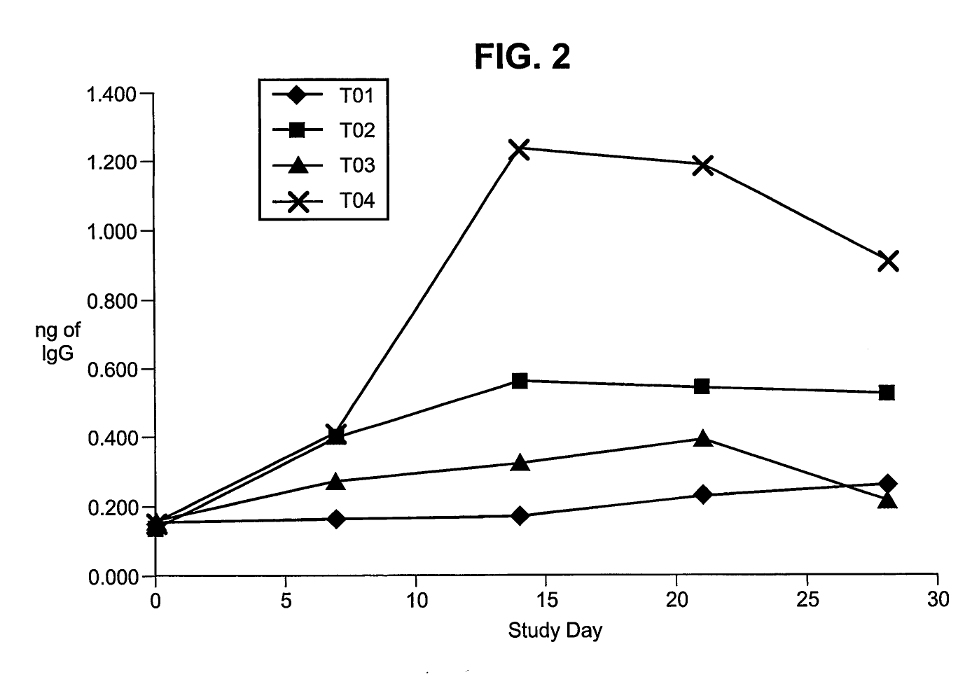

Anti-whole cell antibodies were also monitored (Table 3 and FIG. 2). On Days 7 and 14, the T02 and T04 mean anti-whole cell antibody levels in ng of IgG were significantly greater compared to T01 (P≦0.05). The mean antibody levels for T04 remained significantly higher than the T01 means for the rest of the study (P≦0.05). On days 14 and 21, the T04 mean antibody levels were significantly higher than those from T02 (P≦0.05). As observed with the anti-leukotoxin antibody levels, the difference between the T02 and T04 whole cell antibody levels decreased during the rest of the study. The mean antibody lees in T01 increased slightly during the study. The whole cell antibody levels in T03 were not significantly different from the T01 levels on any of the sample days (P>0.05).

TABLE 2
Anti-leukotoxin geometric mean antibody titer for each treatment
group.

| Treatment | Study Day | | | | |
|---|---|---|---|---|---|
| Group | 0 | 7 | 14 | 21 | 28 |
| T01 | 0.122^{a} | 0.141^{a} | 0.127^{a} | 0.263^{a} | 0.201^{a} |
| T02 | 0.131^{a} | 0.573^{b} | 0.892^{b} | 0.778^{b} | 0.701^{b} |
| T03 | 0.101^{a} | 0.263^{ab} | 0.388^{a} | 0.468^{ab} | 0.293^{a} |
| T04 | 0.114^{a} | 0.479^{b} | 1.542^{c} | 1.382^{c} | 1.078^{b} |

Means within columns with different superscripts are significantly different (P ≦ 0.05).

TABLE 3
Anti-whole cell geometric mean antibody titer for each treatment group.

| Treatment | Study Day | | | | |
|---|---|---|---|---|---|
| Group | 0 | 7 | 14 | 21 | 28 |
| T01 | 0.149^{a} | 0.154^{a} | 0.164^{a} | 0.222^{a} | 0.249^{a} |
| T02 | 0.125^{a} | 0.399^{b} | 0.562^{b} | 0.541^{a} | 0.524^{ab} |
| T03 | 0.158^{a} | 0.262^{ab} | 0.320^{ab} | 0.382^{a} | 0.208^{a} |
| T04 | 0.151^{a} | 0.413^{b} | 1.236^{c} | 1.180^{b} | 0.907^{b} |

Means in a column with different superscripts are significantly different (P ≦ 0.05)

### EXAMPLE 3

### Post-Challenge Clinical Observations

For the vaccination challenge, 5 ml of a virulent culture of M. haemolytica (Oklahoma State strain) was administered by transthoracic injection into the right and left caudal lung lobes of each calf (10 ml of culture per calf) on Day 34 of the study. The inoculum contained approximately 5.6×10⁸ CFU/ml.

Clinical scores (Appendix 2) were assessed prior to challenge on Day 33 and once daily for the duration of the study. These scores reflected an assessment of attitude and respiratory effort. The least squares mean percentage of post-challenge days with at least one clinical score >0 for each of the assessments is summarized in Table 4. The mean percentages were not different in the groups for attitude although T04 was the lowest. (P>0.05). The percentage of days with respiratory effort scores of >0 was significantly less in T04 when compared with the other groups (P≦0.05).

TABLE 4
Least squares mean percentage of post-challenge
days with a clinical score >0 by clinical sign.

| | |
|---|---|
| Treatment | Clinical Sign % of Days |

| Group | Attitude | Respiratory Effort |
|---|---|---|
| T01 | 63.1 | 48.2^{a} |
| T02 | 56.4 | 45.8^{a} |
| T03 | 52.7 | 48.9^{a} |
| T04 | 41.6 | 19.4^{b} |

Means in a column with different superscripts are significantly different (P ≦ 0.05).

The mean percentage of lung consolidation for each group is summarized in Table 5. One animal in T01 died immediately following challenge due to pulmonary hemorrhage as a result of challenge administration; thus its lung lesion data was excluded from the analysis. One animal in T03 was found dead on Day 37 as a result of severe pneumonia but was necropsied and its lung data was analyzed along with the data from the other animals. There were no significant differences between the treatment groups (P>0.05). Both T02 and T04 had fewer lung lesions compared with the control while T03 had increased lesions.

TABLE 5
Least squares mean of percentage of lung lesions.

| Treatment | | Least Squares Mean | Range of Lung |
|---|---|---|---|
| Group | n | Percentage Lung Lesions | Lesion Percentage |
| T01 | 9 | 12.8 | 3.6-30.0 |
| T02 | 10 | 10.0 | 4.6-32.8 |
| T03 | 10 | 21.2 | 8.8-61.8 |
| T04 | 10 | 9.1 | 2.5-31.5 |

Following challenge, animals in all groups showed typical symptoms of respiratory disease. The groups receiving complete vaccine or antigen plus tulathromycin had fewer lung lesions compared to the control group. The group receiving just the antigen without adjuvant had increased lung lesions compared with the other groups. Tulathromycin appeared to effectively replace the adjuvant in One Shot® vaccine, demonstrating the adjuvant function of tulathromycin.

TABLE 6
Appendix
Clinical Scoring System

| Clinical Evaluation | Clinical Scores |
|---|---|
| Attitude | 0 = Normal. Alert, active, stands, moves and responds to stimuli |
| | quickly and steadily, shows continuous interest in |
| | surroundings. |
| | 1 = Mild. Lethargic and somnolent, stands, moves and responds to |
| | stimuli slowly and unsteadily, holds head low, lies down |
| | occasionally. |
| | 2 = Moderate. Tends to lie down frequently, lethargic and |
| | somnolent, stands, moves and responds to stimuli reluctantly |
| | and unsteadily, holds head low, staggers, shows little interest in |
| | surroundings. |
| | 3 = Severe. Recumbent or shows little or no response to stimuli or |
| | stands/moves with difficulty. Animal should be euthanized |
| | for humane reasons. |
| Respiratory effort | 0 = Normal. Respirations are shallow and mostly thoracic |
| | (difficult to see at a distance of approximately 10 feet). |
| | 1 = Slight. Respirations are deep and largely abdominal (easy to |
| | see at a distance of approximately 10 feet). |
| | 2 = Marked. Respirations are labored and entirely abdominal. |
| | 3 = Severe. Respirations are very labored or animal grunts during |
| | breathing. Animal should be euthanized for humane reasons. |

### CONCLUSION

As illustrated by Examples 1-4, T04 was as good if not better than T02 as exhibited by higher antibody production, better attitude and respiratory effect, and fewer lung lesions—all of which are indicators of an immune response to an antigen. Further confirmation of adjuvant properties of tulathromycin is illustrated by comparing T04 results against T03 results. Still further confirmation can be found in T01 and T03 antibody results, which indicate that over the same amount of time (compared to T02 and T04), there was little to no change in antibody production.

### EXAMPLE 5

### Azalide Preparation

One thousand liters of an injectable pharmaceutical composition containing 100 mg of an equilibrium mixture of compounds I and II per mL of composition were prepared as follows.

Approximately 400 liters of Water for Injections (United States Pharmacopeia (USP)/Pharmacopoeia Europa (Ph. Eur.) grade) was added to a stainless steel compounding vessel. Nitrogen (United States National Formulary (NF)/Ph. Eur. grade) was bubbled through the water and agitation was begun. Nitrogen (NF/Ph. Eur. grade) was also used as an overlay to reduce oxygen exposure of the solution in the compound vessel throughout manufacture. The solution was agitated throughout manufacture except during the final sampling and volume check. 19.2 kg of anhydrous citric acid (USP/Ph. Eur. grade) was added to the water. The resulting mixture was agitated until the acid dissolved. 7.8 kg of concentrated hydrochloric acid (NF/Ph. Eur. grade), was added to the mixture and dispersed. 103.0 kg of a mixture containing approximately 97% of compound I and compound II in a ratio exceeding 99:1 and approximately 3% of one or more impurities was added to the agitating mixture over a period of approximately one hour. The total amount of compound I and compound II added to the solution was 100.0 kg. The formulation was agitated until dissolution of the mixture of compound I, compound II, and the one or more impurities appeared complete. Agitation was continued for approximately one hour after dissolution appeared complete. The pH of the resulting solution was adjusted to 7.0±0.3 by adding a total of 0.25 kg of concentrated hydrochloric acid (NF/Ph. Eur. grade) in multiple portions. Equilibration of compound I and compound II was achieved at elevated temperature. The temperature of the solution was raised to 60±3° C. which took approximately 15 minutes. The solution was held at 60±3° C. for approximately 120 minutes. At the end of this period, the ratio of compound I to compound II was approximately 90:10 as determined by HPLC. The solution was then cooled to approximately 25° C. which took approximately 45 minutes. 500 kg of propylene glycol (USP/Ph. Eur. grade) was added to the solution and dispersed. Nitrogen (NF/Ph. Eur. grade) was bubbled through the solution. 5.0 kg of monothioglycerol (NF grade) was added to the solution and dispersed. 10.5 kg of concentrated hydrochloric acid (NF/Ph. Eur. grade), was added to the mixture and dispersed. The pH of the solution was adjusted to 5.4±0.3 by addition of approximately 0.85 kg of concentrated hydrochloric acid (NF/Ph. Eur. grade) in multiple portions. Sufficient Water for Injections (USP/Ph. Eur. grade) was added to produce a final volume of 1000 liters. The resulting composition contained 100 mg of an equilibrated mixture of compounds I and II per mL of composition, 500 mg of propylene glycol per mL of the composition, 5.0 mg of monothioglycerol per mL of the composition, and 19.2 mg (0.100 millimole) of citric acid per mL of the composition.

The composition was filtered through 0.2 micron Millipore Milligard (Millipore Corporation, Billerica, Mass., USA) pre-filter into a stainless steel receiving tank and held for approximately 60 hours. The composition was sterilized by filtering it through redundant 0.2 micron Millipore Durapore (Millipore S A, Molsheim France) sterilizing filters. The sterilizing filters were sterilized by moist heat autoclaving for 45 minutes at 122° C. The filters were tested for integrity using both bubble point and diffusion test methods prior to their sterilization and after being used for filtration of the solution. 20 mL flint, type I glass serum vials (Saint Gobain des Jonqueres, Mers les Bains, France) were sterilized and depyrogenated in a dry heat tunnel with a set point of 350° C. The minimum exposure time was 31 minutes. 20 mm chlorobutyl. rubber stoppers coated with Daikyo Fluoro Resin-D (Daikyo-Seiko, Tokyo, Japan) were depyrogenated by washing and sterilized by moist-heat autoclaving for 60 minutes at 124° C. Each of 1444 of the 20 mL vials was filled under sterile conditions with 20.6 mL of the resulting composition. Each vial contained 2.06 g of an equilibrated mixture of compounds I and II. The vial headspace was flushed with nitrogen and the vials were sealed with the stoppers and appropriate aluminum overseals (Helvoet Pharma, Alken, Belgium). 500 mL flint, type I glass serum vials (Saint Gobain des Jonqueres, Mers les Bains, France) were sterilized and depyrogenated in a dry heat tunnel with a set point of 350° C. The minimum exposure time was 38 minutes. 32 mm chlorobutyl rubber stoppers coated with Daikyo Fluoro Resin-D (Daikyo-Seiko, Tokyo, Japan) were depyrogenated by washing and sterilized by moist-heat autoclaving for 60 minutes at 124° C. Each of 1537 of the 500 mL vials was filled under sterile conditions with 510 mL of the resulting composition. Each vial contained 51.0 g of an equilibrated mixture of compounds I and II. The vial headspace was flushed with nitrogen and the vials were sealed with the stoppers and appropriate aluminum overseals (Helvoet Pharma, Alken, Belgium).

### Additional Antimicrobial Agents

In addition to the examples provided above numerous other antimicrobial agents are suitable for use as the antimicrobial agent component of this invention and are hereby described below with particularity. For the agents described below the amount of agent and the duration of its administration can be easily determined. Antimicrobial agents with short periods of effectiveness will typically need to be given more frequently and with a longer duration. Antimicrobial agents with a longer half life may be administered less frequently. Dosage range provided below both for animals and humans will provide guidance as to the effective dose for an adjuvant. Both gram-positive and gram-negative antibiotic agents are included in this description.

Anti-Microbial Agents typically directed to non-human animals:

TABLE 7
Penam Penicillins:

| | | | | Interval |
|---|---|---|---|---|
| Drug | Dose (IU/kg or mg/kg) | | Route | (h) |
| Penicillin G, | 15,000-20000 | IU/kg | IM, IV | 6-8 |
| sodium aqueous | | | | |
| Procaine | 25,000 | IU/kg | IM | 24 |
| penicillin G | | | | |
| Benzathine | 40,000 | IU/kg | IM | 72 |
| penicillin | | | | |
| Penicillin V | 10 | mg/kg | Oral | 6-8 |
| Cloxacillin, | 15-25 | mg/kg | Oral | 6-8 |
| dicloxacilling, | | | | |
| methicillin, | | | | |
| oxacilling | | | | |
| Ampicillin sodium | 10-20 | mg/kg | IM, IV | 6-8 |
| Ampicillin | 10-20 | mg/kg | Oral | 8 |
| (hetacilling) | | | | |
| Amoxicillin | 10-20 | mg/kg | Oral | 8-12 |
| Amoxicillin | 10 | mg/kg | IM (SC) | 12 |
| Amoxicillin | 15 | mg/kg | IM | 48 |
| long-acting | | | | |
| Amoxicillin | 10-20 | mg/kg | IM | 12 |
| trihydrate | | | | |
| Pivampicillin | 25 | mg/kg | Oral | 12 |
| Carbenicillin, | 33 | mg/kg | Oral | 6-8 |
| indanyl sodium | | | | |
| Carbenicillin | 33 | mg/kg | IM, IV | 6-8 |
| Piperacillin | 50 | mg/kg | IV (IM) | 8 |
| Ticarcillin | 25-40 | mg/kg | IV (IM, | 8 |
| | | | SC) | |
| Ureidopenicillin | | | | |
| Tricarcillin | | | | |
| Dzlocillin | | | | |
| Temocillin | | | | |
| Nafcillin | | | | |
| Aminobenzylpenicillius | | | | |
| Mecillinam | | | | |
| Carboxypenicillin | | | | |

TABLE 8
Beta-lactam antibiotics

| | | | | |
|---|---|---|---|---|
| Drug | Dose (mg/kg) | Species | Route | Interval (h) |

Cephalosporins - Cephalosporins, Parenteral dosage (IV, IM, SC).

| | | | |
|---|---|---|---|
| Cephradine | 22 | dogs, cats | 6-8 |
| Cephalothin | 20-40 | dogs, cats | 6-8 |
| Cefazolin | 15-30 | dogs, cats | 12 |
| Cephapirin | 20 | horse | 8 |
| Cefazolin | 15-20 | horse | 8 |
| Cephalexin | 10 | horse | 8-12 |
| Cefazolin | 15-20 | cattle, sheep | 12 |
| Cephapirin | 10 | cattle, sheep | 8-12 |

Oral
Cephalosporins

| | | | |
|---|---|---|---|
| Cefadroxil | 22 | dogs, cats | 12 |
| Others | 10-15 | dogs, cats | 8 |
| Cefadroxil | 25 | calves | 12 |
| | | (preruminants) | |
| Cefaclor | 3.5 | calves | 12 |
| | | (preruminants) | |
| Cephadrine | 7 | calves | 12 |
| | | (preruminants) | |
| Cefadroxil | 20-40 | horses | 8 |

Other
Parenteral Cephalosporins

| | | | | |
|---|---|---|---|---|
| Cefotaxime | 20-40 | Dogs, cats | IM | 8 |
| Cefotaxime | 20-40 | Dogs, cats | SC | 12 |
| Cefoperazone | 20-25 | Dogs, cats | IV, IM | 6-8 |
| Cefoxitin | 15-30 | Dogs, cats | IV, IM, | 6-8 |
| | | | SC | |
| Ceftiofur | 2.2 | Dogs, cats | IM | 24 |
| Ceftizoxime | 25-40 | Dogs, cats | IV, IM | 8-12 |
| Ceftriaxone | 25 | Dogs, cats | IV, IM | 12-24 |
| Cefuroxime | 10-15 | Dogs, cats | PO | 8-12 |
| axetil | | | | |
| Cefuroxime | 10-15 | Dogs, cats | IV | 8-12 |
| Ceftiofur | 1-2.2 | Cattle | IM | 24 |
| Cefquinome | 1 | Cattle | IM | 24 |
| Cefotaxime | 20-40 | Goats | IV, IM | 12 |
| Cefotaxime | 20-30 | Horses | IV | 6-8 |
| Cefoxitin | 20 | Horses | IV, IM | 8 |
| Ceftiofur | 2.2 | Horses | IM | 12-24 |
| Ceftriaxone | 25 | Horses | IV, IM | 12 (not |
| | | | | adults?) |
| Ceftiofur | 2.2 | Swine | IM | 24 |

Antipseudomonal parenteral cephalosporins

| | | | | |
|---|---|---|---|---|
| Cefoperazone | 30 | Dogs, cats | IM | 6-8 |
| Ceftazidime | 25-50 | Dogs, cats | IM | 8-12 |
| Cefoperazone | 30 | Cattle | IM | 6-8 |
| Ceftazidime | 20-40 | Cattle | IM | 12-24 |
| Cefoperazone | 30 | Horses | IM | 6-8 |
| | | (caution) | | |
| Ceftazidime | 25-50 | Horses | IM | 8-12 |
| | | (caution) | | |

Penicillins potentiated by clavulanic acid, sulbactam, tazobactam

| | | | | |
|---|---|---|---|---|
| Clavulanate- | 12.5-20 | Dogs, cats | PO | 8-12 |
| Amoxicillin | 10 | Dog, cats | SC | 8 |
| | 7 | Cattle | IM | 12-24 |
| | 5-10 | Preruminant | PO | 12 |
| | 8.75 | Sheep | IM | 12-24 |
| Clavulanate- | 40-50 | Dog, cats | IV | 6-8 |
| ticarcillin | 50 | Horses | IV | 6 |
| Sulbactam- | 10 | Cattle | IM | 24 |
| ampicillin | | | | |
| Piperacillin- | 4 | Dogs, cats | IV | 6 |
| tazobactam | | | | |

TABLE 9
Aminoglycosides and aminocyclitols

| | | | | Interval |
|---|---|---|---|---|
| Drug | Dose (mg/kg) | Species | Route | (h) |
| Amikacin^{b} | 21 | Horses | IM (IV)^{b} | 24 |
| | 15-20 | Dogs, cats | IM, SC | 24 |
| Apramycin | 20 | Enteric infection | PO | 12 |
| | 20 | Cattle, swine only | IM | 24 |
| Gentamicin | 7-10 | | IM, SC (IV) | 24 |
| Kanamycin | 10 | Enteric infection | PO | 6 |
| | 18 | | IM, SC | 24 |
| Neomycin | 10 | Enteric infection | PO | 6 |
| Spectomycin | 20-40 | Enteric infection | PO | 8 |
| | 20-30 | Calves, pigs | IM, SC | 12 |
| Streptomycin | 20 | | IM | 24 |
| Tobramycin | 6 | | IM, SC (IV) | 24 |

### Table 10

Lincosamides, Pleuromutilium, Chloramphenicols and Macrolides
Lincosamides-lincomycine, Clindamycin and Pirlimycine
Pleuromutilins—Tiamulin, Valnemulin
Chloramphenicol, Thiaphenicol, and Florfenical.
Macrolides—Erythromycin, Tylasin, Spiramycin, Tilmicosin, Roxithromycin,

Azithromycin, Clarithromycin, Ketolide, and Tulathromycin also described above.

TABLE 11
Tetracyclines in animals.

| | Dose | | | Interval |
|---|---|---|---|---|
| Drug | (mg/kg) | Species | Route | (h) |
| Tetracycline | 10 | Dogs | IV, IM | 12 |
| oxytetracycline | | and Cats | | |
| Doxycycline | 5-10 | | IV (not IM) | 12 |
| Tetracycline | 10 | Horses | IV | 12 |
| Oxytetracycline | 3-5 | | IV | 12 |
| Tetracycline, | 10 | Ruminants | IV, IM | 12-24 |
| oxytetracycline | | | | |
| Long-acting | 20 | | IM | 48 |
| tetracycline | | | | |
| Tetracycline, | 10-20 | Pigs | IM | 12-24 |
| oxytetracycline | | | | |
| Long-acting | 20 | | IM | 48 |
| tetracycline | | | | |
| Tetracycline HCl | 15 | Swine 200-800 ppm | | 6-8 |
| Oxytetracycline | 20 | | | 8-12 |
| HCl | | | | |
| Minocycline HCl | | | | 12 |
| Doxycycline | 5 | Swine 200-250 ppm | | 12 |
| hyclate | | | | |

TABLE 12
Sulfonamides in animals

| | Dose | | | |
|---|---|---|---|---|
| Drug | (mg/kg) | Species | Route | Interval (h) |
| Short-acting sulfadiazine, | 50-60 | | IV, PO | 12 |
| sulfamethazine, | | | | |
| trisulfapyrimidine | | | | |
| (triple sulfas) | | | | |
| Sulfamethoxazole | 50 | | PO | 12 |
| Intermediate-acting | 27.5 | | PO, IV, | 24 |
| | | | IM, SC | |
| sulfadimethoxine | 137.5 | | PO | 96 |
| (sustained release, cattle) | 50 | Cattle | PO, IV | 12 |
| sulfadiazine | | | | |
| Sulfisoxazole | 50 | | PO | 8 |
| phthalylsulfathiazole | 100 | | PO | 12 |
| | | | (Gut-active) | |
| Special-use | | | | |
| salicylazolsulfapyridine | 25 | | PO | 12 |
| silver sulfadiazine | | | Topical | |

TABLE 13
Fluoroquinolones

| | |
|---|---|
| Enrofloxacin | Dogs, cats, chickens, turkeys, beef cattle, horses, pigs |
| Orbifloxacin | Dogs, cats |
| Difloxacin | Dogs, chickens, turkeys |
| Danofloxacin | Cattle, pigs |
| Marbofloxacin | Dogs, cats, pigs, cattle |
| Sarafloxacin | Chichens, turkeys |

### Antimicrobial Agents Typically Directed to Humans

### Table 14

Specifically, we disclose Amikacin, Gentamicin, Spectinomycin, Tobramycin, Imipenem, Meropenem, Cefadroxil, Cefazolin, Cephalexin, Cefaclor, Cefotetan, Cefoxitin, Cefprozil, Cefuroxime, Loracarbef, Cefdinir, Cefixime, Cefoperazone, Cefotaxime, Cefpodoxime, Ceftazidime, Ceftibuten, Ceftozoxime, Ceftriaxone, Cefepime, Azithromycin, Clarithromycin, Dirithromycin, Penicillin G, Cloxacillin, Dicloxacillin, Nafcillin, Oxacillin, Amoxicillin, Amoxicillin, Ampicillin, Mezlocillin, Piperacillin, Nalidixic Acid, Ciprofloxacin, Enoxacin, Lomefloxacin, Norfloxacin, Ofloxacin, Levofloxacin, Sparfloxacin, Alatrofloxacin, Gatifloxacin, Moxifloxacin, Trimethoprim, Sulfisoxazole, Sulfamethoxazole, Doxycycline, Minocycline, Tetracycline, Aztreonam, Chloramphenicol, Clindamycin, Quinupristin, Fosfomycin, Metronidazole, Nitrofurantoin, Rifampin, Trimethoprim, and Vancomycin. All of these are known. They can be either obtained commercially or be prepared according to the references cited in PHYSICIANS' DESK REFERENCE, the 53^{rd} Edition (1999) and the US FDA's Orange book.

The term “gram-positive antibiotic” refers to an antibacterial agent active against gram-positive bacterial organisms. The term “gram-negative antibiotic” refers to an antibacterial agent active against gram-negative bacterial organisms.

TABLE 15
Gram-positive antibiotics that may be used
in a combination therapy with the compound of formula I

| AGENTS | LO DOSE | HI DOSE | STD DOSE |
|---|---|---|---|
| AMINO- | | | |
| GLYCOSIDES | | | |
| Amikacin | | | 15 mg/kg/day |
| Gentamicin | 1 mg/kg/day | 5 mg/kg/day | |
| | .5 mg/kg | 2.5 mg/kg | |
| Spectinomycin | | | 40 mg/kg |
| Tobramycin | 1 mg/kg/day | 5 mg/kg/day | |
| | .5 mg/kg/day | 5 mg/kg/day | |
| PENEMS | | | |
| Imipenem/cilastatin | 62.5 mg | 1 g | |
| | 6.25 mg/kg | 25 mg/kg | |
| Meropenem | | | 40 mg/kg |
| | .5 mg/kg | 2.5 mg/kg | |
| 1^{ST} GEN CEPHS | | | |
| Cefadroxil | .25 g/day | 2 g/day | |
| | | | 30 mg/kg/day |
| Cefazolin | 62.5 mg | 1.5 g | |
| | 6.25 mg/kg/day | 100 mg/kg/day | |
| Cephalexin | 62.5 mg | 500 mg | |
| | 6.25 mg/kg/day | 50 mg/kg/day | |
| 2^{ND} GEN CEPHS | | | |
| Cefaclor | 62.5 mg | 500 mg | |
| | 5 mg/kg/day | 40 mg/kg/day | |
| Cefotetan | 0.125 g | 3 g | |
| | 10 mg/kg/day | 80 mg/kg/day | |
| Cefoxitin | .25 g | 3 g | |
| | 20 mg/kg/day | 160 mg/kg/day | |
| Cefprozil | 62.5 mg | 500 mg | |
| | 1.87 mg/kg/dose | 15 mg/kg/dose | |
| Cefuroxime | 187.5 mg | 3 g | |
| | 31.25 mg | 500 mg | |
| | 12.5 mg/kg/day | 150 mg/kg/day | |
| | 31.25 mg/kg/day | 500 mg/kg/day | |
| Loracarbef | 50 mg | 400 mg | |
| | 3.75 mg/kg/day | 500 mg/kg/day | |
| 3^{RD} GEN CEPHS | | | |
| Cefdinir | 75 mg | | 600 mg |
| Cefixime | 50 mg | | 400 mg |
| Cefoperazone | .5 g/day | 12 g/day | |
| | 25 mg/kg/day | 150 mg/kg/day | |
| Cefotaxime | .25 g | 2 g | |
| | 12.5 mg/kg/dose | 300 mg/kg/day | |
| Cefpodoxime | 25 mg | 400 mg | 10 mg/kg/day |
| Ceftazidime | 62.5 mg | 2 g q8 | |
| | 25 mg/kg/day | 150 mg/kg/day | |
| Ceftibuten | 2.25 mg/kg | 400 mg | 400 mg |
| Ceftozoxime | .25 g | 4 g | |
| | 12.5 mg/kg/day | 200 mg/kg/day | |
| Ceftriaxone | 31.25 mg | 2 g | |
| | 12.5 mg/kg/day | 100 mg/kg/day | |
| 4^{TH} GEN CEPHS | | | |
| Cefepime | 0.125 g | 2 g | |
| | 12.5 mg/kg | 50 mg/kg q8 | |
| MACROLIDES | | | |
| Azithromycin | 62.5 mg | 500 mg | |
| | 62.5 mg | 500 mg | |
| Clarithromycin | 62.5 mg | 500 mg | 7.5 mg/kg/day |
| Dirithromycin | | | 500 mg |
| 1^{ST} GEN PENS | | | |
| Penicillin G | 2 million | 30 million | |
| | units/day | units/day | |
| | 2000units/kg/dy | 400,000 | |
| | | units/kg/day | |
| 2^{ND} GEN PENS | | | |
| Cloxacillin | 62.5 mg | 500 mg | |
| | 12.5 mg/kg/day | 100 mg/kg/day | |
| Dicloxacillin | 31.25 mg | 500 mg | |
| | 3.125 mg/kg/day | 100 mg/kg/day | |
| Nafcillin | 125 mg | 2 g | |
| | 2.5 mg/kg | 25 mg/kg | |
| Oxacillin | 62.5 mg | 2 g | |
| | 125 mg | 1000 mg | |
| | 25 mg/kg/day | 200 mg/kg/day | |
| | 12.5 mg/kg/day | 100 mg/kg/day | |
| 3^{RD} GEN PENS | | | |
| Amoxicillin | 62.5 mg | 875 mg | |
| | 5 mg/kg/day | 45 mg/kg | |
| Amoxicillin/ | 62.5 mg | 875 mg | |
| clavulanic acid | 6.25 mg/kg/day | 45 mg/kg/day | |
| Ampicillin | 62.5 mg | 12 g/day q4 | |
| | 6.25 mg/kg/day | 300 mg/kg/day | |
| Ampicillin/ | 0.375 g | 3 g | 300 mg/kg/day |
| sulbactam | | | |
| 4^{TH} GEN PENS | | | |
| Mezlocillin | 0.375 g | 4 g | 75 mg/kg |
| Piperacillin | 1.5 g/day | 24 g day | |
| | 25 mg/kg/day | 300 mg/kg/day | |
| Piperacillin/ | | | 240 mg/kg/day |
| tazobactam | | | |
| Ticarcillin | .25 g | 4 g | |
| | 12.5 mg/kg/day | 300 mg/kg/day | |
| Ticarcillin/ | 50 mg/kg/day | 300 mg/kg/day | |
| clavulanate | 0.775 g | 3.1 g | |
| 1^{ST} GEN | | | |
| QUINOLONES | | | |
| Nalidixic Acid | | | 55 mg/kg/day |
| 2^{ND} GEN | | | |
| QUINOLONES | | | |
| Ciprofloxacin | 50 mg | 750 mg | |
| | 2.5 mg/kg/dose | 15 mg/kg/dose | |
| | 62.5 mg | 750 mg | |
| | 2.5 mg/kg/dose | 15 mg/kg/dose | |
| Enoxacin | 50 mg | 400 mg | |
| Lomefloxacin | | | 400 mg |
| Norfloxacin | | | 400 mg |
| Ofloxacin | 50 mg | 400 mg | |
| 3^{RD} GEN | | | |
| QUINOLONES | | | |
| Levofloxacin | 62.5 mg | 750 mg | |
| Sparfloxacin | 50 mg | 400 mg | |
| 4^{TH} GEN | | | |
| QUINOLONES | | | |
| Alatrofloxacin | 50 mg | 300 mg | |
| Gatifloxacin | 50 mg | 400 mg | |
| Moxifloxacin | | | 400 mg |
| SULFAS | | | |
| Trimethoprim/ | 15 mg | 800 mg | |
| sulfamethoxazole | 3.75 mg/day | 150 mg/day | |
| Sulfisoxazole | 18.75 mg | 150 mg | |
| Sulfamethoxazole | .25 g | 2 g | |
| TETRACYCLINES | | | |
| Doxycycline | 5 mg | 100 mg | |
| Minocycline | 25 mg | 200 mg | |
| Tetracycline | 62.5 mg | 500 mg | |
| OTHER | | | |
| Chloramphenicol | 12.5 mg/kg/day | 100 mg/kg/day | |
| Clindamycin | 150 mg | 900 mg | |
| | 37.5 mg | 450 mg | |
| | 5 mg/kg/day | 40 mg/kg/day | |
| | 2 mg/kg/day | 25 mg/kg/day | |
| Quinupristin/ | 1.875 mg/kg | 7.5 mg/kg q8 | |
| dalfopristin | | | |
| Fosfomycin | | | 3 g |
| Nitrofurantoin | 12.5 mg | 100 mg | |
| | 1.25 mg/kg/day | 7 mg/kg/day | |
| Rifampin | 2.5 mg/kg | 600 mg/kg | |
| | 2.5 mg/kg | 600 mg/kg | |
| Trimethoprim | 25 mg | 200 mg | 10 mg/kg/day |
| Vancomycin | | | 1 g |
| | 2.5 mg/kg q6 | 15 mg/kg q8 | |

In combating the infective diseases caused by gram-positive and gram-negative organisms, the compound of the formula I can be used in combination with other antibiotics that are active against gram-negative organisms. Examples of such gram-negative antibiotics are listed in Table 2. Some of gram-negative antibiotics may also have activity against gram-positive organisms.

TABLE 16
Gram-Negative Antibiotics

| AGENTS | LO DOSE | HI DOSE | STD DOSE |
|---|---|---|---|
| AMINO- | | | |
| GLYCOSIDES | | | |
| Amikacin | | | 15 mg/kg/ |
| | | | day |
| Gentamicin | 0.75 mg/kg/day | 5 mg/kg/day | |
| | 0.5 mg/kg | 2.5 mg/kg | |
| Spectinomycin | | | 40 mg/kg |
| Tobramycin | 0.75 mg/kg/day | 5 mg/kg/day | |
| | 0.5 mg/kg/day | 5 mg/kg/day | |
| PENEMS | | | |
| Imipenem/cilastatin | 62.5 mg | 1 g | |
| | 6.25 mg/kg | 25 mg/kg | |
| Meropenem | | | 40 mg/kg |
| | 0.5 mg/kg | 2.5 mg/kg | |
| 2^{ND} GEN CEPHS | | | |
| Cefaclor | 62.5 mg | 500 mg | |
| | 5 mg/kg/day | 40 mg/kg/day | |
| Cefotetan | 0.125 g | 3 g | |
| | 10 mg/kg/day | 80 mg/kg/day | |
| Cefoxitin | 0.25 g | 3 g | |
| | 20 mg/kg/day | 160 mg/kg/day | |
| Cefprozil | 62.5 mg | 500 mg | |
| | 1.875 mg/kg/ | 15 mg/kg/dose | |
| | dose | | |
| Cefuroxime | 187.5 mg | 3 g | |
| | 31.25 mg | 500 mg | |
| | 12.5 mg/kg/day | 150 mg/kg/day | |
| | 31.25 mg/kg/day | 500 mg/kg/day | |
| Loracarbef | 50 mg | 400 mg | |
| | 3.75 mg/kg/day | 500 mg/kg/day | |
| 3^{RD} GEN CEPHS | | | |
| Cefdinir | 75 mg | | 600 mg qd |
| Cefixime | 50 mg | | 400 mg |
| Cefoperazone | 0.25 g/day | 12 g/day | |
| | 25 mg/kg/day | 150 mg/kg/day | |
| Cefotaxime | 0.25 g | 2 g | |
| | 12.5 mg/kg/dose | 300 mg/kg/day | |
| Cefpodoxime | 25 mg | 400 mg | 10 mg/kg/ |
| | | | day |
| Ceftazidime | 62.5 mg | 2 g q8 | |
| | 25 mg/kg/day | 150 mg/kg/day | |
| Ceftibuten | 2.25 mg/kg | 400 mg | 400 mg |
| Ceftozoxime | 0.25 g | 4 g | |
| | 12.5 mg/kg/day | 200 mg/kg/day | |
| Ceftriaxone | 31.25 mg | 2 g | |
| | 12.5 mg/kg/day | 100 mg/kg/day | |
| 4^{TH} GEN CEPHS | | | |
| Cefepime | 0.125 g | 2 g | |
| | 12.5 mg/kg | 50 mg/kg q8 | |
| MACROLIDES | | | |
| Azithromycin | 62.5 mg | 500 mg | |
| | 62.5 mg | 500 mg | |
| Clarithromycin | 62.5 mg | 500 mg | 7.5 mg/kg/ |
| | | | day |
| Dirithromycin | | | 500 mg |
| 3^{RD} GEN PENS | | | |
| Amoxicillin | 62.5 mg | 875 mg | |
| | 5 mg/kg/day | 45 mg/kg | |
| Amoxicillin/ | 62.5 mg | 875 mg | |
| clavulanic acid | 6.25 mg/kg/day | 45 mg/kg/day | |
| Ampicillin | 62.5 mg | 12 g/day q4 | |
| | 6.25 mg/kg/day | 300 mg/kg/day | |
| Ampicillin/sulbactam | 0.375 g | 3 g | 300 mg/kg/ |
| | | | day |
| 4^{TH} GEN PENS | | | |
| Mezlocillin | 0.375 g | 4 g | 75 mg/kg |
| Piperacillin | 1.5 g/day | 24 g day | |
| | 25 mg/kg/day | 300 mg/kg/day | |
| Piperacillin/ | | | 240 mg/kg/ |
| tazobactam | | | day |
| Ticarcillin | 0.25 g | 4 g | |
| | 12.5 mg/kg/day | 300 mg/kg/day | |
| Ticarcillin/clavulanate | 50 mg/kg/day | 300 mg/kg/day | |
| | 0.775 g | 3.1 g | |
| 1^{ST} GEN | | | |
| QUINOLONES | | | |
| Nalidixic Acid | | | 55 mg/kg/ |
| | | | day |
| 2^{ND} GEN | | | |
| QUINOLONES | | | |
| Ciprofloxacin | 50 mg | 750 mg | |
| | 2.5 mg/kg/dose | 15 mg/kg/dose | |
| | 62.5 mg | 750 mg | |
| | 2.5 mg/kg/dose | 15 mg/kg/dose | |
| Enoxacin | 50 mg | 400 mg | |
| Lomefloxacin | | | 400 mg |
| Norfloxacin | | | 400 mg |
| Ofloxacin | 50 mg | 400 mg | |
| 3^{RD} GEN | | | |
| QUINOLONES | | | |
| Levofloxacin | 62.5 mg | 750 mg | |
| Sparfloxacin | 50 mg | 400 mg | |
| 4^{TH} GEN | | | |
| QUINOLONES | | | |
| Alatrofloxacin | 50 mg | 300 mg | |
| Gatifloxacin | 50 mg | 400 mg | |
| Moxifloxacin | | | 400 mg |
| SULFAS | | | |
| Trimethoprim/ | 15/200 mg | | |
| sulfamethoxazole | 3.75 mg/day | 150 mg/day | |
| Sulfisoxazole | 18.75 mg | 150 mg | |
| Sulfamethoxazole | 0.25 g | 2 g | |
| TETRACYCLINES | | | |
| Doxycycline | 5 mg | 100 mg | |
| Minocycline | 25 mg | 200 mg | |
| Tetracycline | 62.5 mg | 500 mg | |
| OTHER | | | |
| Chloramphenicol | 12.5 mg/kg/day | 100 mg/kg/day | |
| Aztreonam | 125 mg | 2 g | |
| | 37.5 mg | 450 mg | |
| | 5 mg/kg/day | 40 mg/kg/day | |
| | 2 mg/kg/day | 25 mg/kg/day | |
| Fosfomycin | | | 3 g |
| Nitrofurantoin | 12.5 mg | 100 mg | |
| | 1.25 mg/kg/day | 7 mg/kg/day | |
| | 2.5 mg/kg | 600 mg/kg | |
| Trimethoprim | 25 mg | 200 mg | 10 mgkgdy |

In Tables 15 and 16, the term “Lo Dose” means the recommended lower dosage for the combination therapy of the invention. It may be adjusted even lower depending on the requirements of each subject being treated and the severity of the bacterial infection. The lowest dosage possible may be 0.1 mg when combined with the compound of formula I of the present invention. The term “Hi Dose” means the recommended highest dosage in the combination therapy. It may be changed hereafter according to the US FDA standard. The term “Std Dose” means the recommended standard dosage for the combination therapy of the present invention. It may be adjusted even lower depending on the requirements of each subject being treated and the severity of the bacterial infection. A specific antibiotic may have more than one the recommended dosage ranges.

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1.-18. (canceled)

19. An adjuvant composition comprising one or more antimicrobial agents.

20. An adjuvant composition of claim 19 for use in a human vaccine.

21. An adjuvant composition of claim 19 for use in a non-human animal vaccine.

22. A human or non-human animal vaccine comprising at least two components, with the two components administered either concurrently, or co-administered within a month, where the first component is an adjuvant comprising one or more antimicrobial agents and the second component is one or more antigenic agents.

23. A vaccine of claim 22 where the antimicrobial agent is a macrolide or beta-lactam antibiotic.

24. A vaccine of claim 22 where the vaccine is for non-human animals, where the antimicrobial agent is selected from the group consisting of tulathromycin and ceftiofur, and where the antigenic agent is one or more antigens selected from one or more from the group consisting of M. haemolytica antigen, M. haemolytica leukotoxin, M. haemolytica capsular antigen, M. haemolytica soluble antigen.

25. An adjuvant composition of claim 19 where said antimicrobial agent is comprises one or more azalides selected from the group consisting of 8a-azalide and a 9a-azalide.

26. An adjuvant composition of claim 19, wherein said azalide is a 9a-azalide of the formula I:

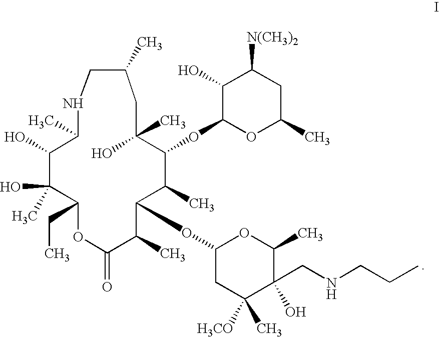

27. An adjuvant composition of claim 22, further comprising a compound of formula II:

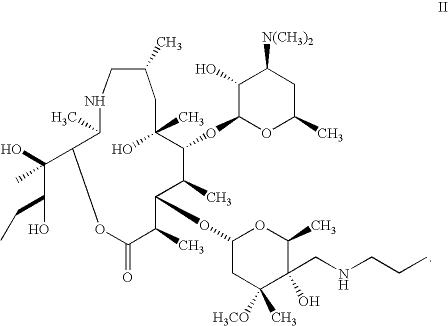

28. An adjuvant composition of claim 27, comprising (a) a mixture of compounds of formulae I and II in a ratio of about 90%±10% to about 10%±10%, respectively, (b) water; and (c) one or more acids present at a total concentration of from about 0.2 mmol to about 1.0 mmol per mL of the composition.

29. A vaccine comprising any of the antimicrobial adjuvant compositions of claim 25 administered either concurrently or co-administered with an antigen.

30. A vaccine comprising the antimicrobial adjuvant compositions of claim 26 administered either concurrently or co-administered with an antigen.

31. A vaccine comprising the antimicrobial adjuvant compositions of claim 27 administered either concurrently or co-administered with an antigen.

32. A vaccine comprising any of the antimicrobial adjuvant compositions of claim 28 administered either concurrently or co-administered with an antigen.

33. A vaccine of claim 29 administered either concurrently or co-administered with an antigen selected from any M. haemolytica antigen with an adjuvant composition of claim 28, wherein said 9a-azalide is a composition comprising (a)(i) a mixture of compounds of formulae I and II in a ratio of about 90%±10% to about 10%±10%, respectively; (ii) water; and (iii) one or more acids present at a total concentration of from about 0.2 mmol to about 1.0 mmol per mL of the composition; and (b) one or more water-miscible co-solvents present in an amount of from about 250 to about 750 mg per mL of the composition.

34. A vaccine of claim 30 administered either concurrently or co-administered with an antigen selected from any M. haemolytica antigen with an adjuvant composition of claim 28, wherein said 9a-azalide is a composition comprising (a)(i) a mixture of compounds of formulae I and II in a ratio of about 90%±10% to about 10%±10%, respectively; (ii) water; and (iii) one or more acids present at a total concentration of from about 0.2 mmol to about 1.0 mmol per mL of the composition; and (b) one or more water-miscible co-solvents present in an amount of from about 250 to about 750 mg per mL of the composition.

35. A vaccine of claim 31 administered either concurrently or co-administered with an antigen selected from any M. haemolytica antigen with an adjuvant composition of claim 28, wherein said 9a-azalide is a composition comprising (a)(i) a mixture of compounds of formulae I and II in a ratio of about 90%±10% to about 10%±10%, respectively; (ii) water; and (iii) one or more acids present at a total concentration of from about 0.2 mmol to about 1.0 mmol per mL of the composition; and (b) one or more water-miscible co-solvents present in an amount of from about 250 to about 750 mg per mL of the composition.

18. A vaccine of claim 32 administered either concurrently or co-administered with an antigen selected from any M. haemolytica antigen with an adjuvant composition of claim 28, wherein said 9a-azalide is a composition comprising (a)(i) a mixture of compounds of formulae I and II in a ratio of about 90%±10% to about 10%±10%, respectively; (ii) water; and (iii) one or more acids present at a total concentration of from about 0.2 mmol to about 1.0 mmol per mL of the composition; and (b) one or more water-miscible co-solvents present in an amount of from about 250 to about 750 mg per mL of the composition.

37. A vaccine administered either concurrently or co-administered with any of the an antigen selected from any M. haemolytica antigen with an adjuvant composition comprising any ceftiofur.

38. A method for enhancing, increasing, upwardly modulating, diversifying or otherwise facilitating an immune response in an animal to an antigen comprising administration of an antimicrobial agent to an animal.

39. A method of claim 38 where the antimicrobial agent is at least one adjuvant component of a concurrent administration of an antimicrobial agents and an antigen, where the antimicrobial agent is selected from the antimicrobial agents; penicillin g, procaine, benzathine, penicillin v, cloxacillin, ampicillin sodium, ampicillin, amoxicillin, pivampicillin, carbenicillin, piperacillin, ticarcillin, ureidopenicillin, dzlocillin, temocillin, nafcilln, aminobenzylpenicillius, mecillinam, carboxypenicillin, cephiradine, cephalothin, cephapirin, cefazolin, cephalexin, cefaclor, cephadrine, cefadroxil, cefoperazone, cefoxitin, ceftiofur, ceftizoxime, ceftriaxone, cefuroxime, cefquinome, cefotaxime, ceftriaxone, ceftazidime, clavulanate-amoxicillin, clavulanate-ticarcillin, sulbactam-ampicillin, piperacillin-tazobactam, amikacin^{b}, aprarycin, gentamicin, kanamycin, neomycin, spectomycin, streptomycin, tobramycin, lincosamides, pleuromutilium, chloramphenicols, macrolides, lincosamides-lincomycine, clindamycin, pirlimycine, pleuromutilins—tiamulin, valnemulin, chloramphenicol, tiaphenicol, florfenical, macrolides—erytromycin, tylasin, spiramycin, tilmicosin, roxithromycin, azithromycin, clarithromycin, ketolide, tulathromycin, oxytetracycline, doxycycline, tetracycline, tetracycline hcl, oxytetracycline hcl minocycline hcl, doxycycline hyclate, sulfamethazine, trisulfapyrimidine, sulfamethoxazole, sulfadimethoxine, sulfadiazine, sulfisoxazole, phthalylsulfathiazole, salicylazolsulfapyridine, silver sulfadiazine, enrofloxacin, orbifloxacin, difloxacin, danofloxacin, marbofloxacin, sarafloxacin, spectinomycin, imipenem, meropenem, cefotetan, cetprozil, loracarbef, cefdinir, cefpodoxime, ceftibuten, ceftozoxime, cefepime, dirithromycin, dicloxacillin, oxacillin, mezlocillin, nalidixic acid, ciprofloxacin, enoxacin, lomefloxacin, norfloxacin, ofloxacin, levofloxacin, spartloxacin, alatrofloxacin, gatifloxacin, moxifloxacin, trimethoprim, aztreonam, quinupristin, fosfomycin, metronidazole, nitrofurantoin, rifampin, vancomycin, (2R,3S,4R,5R,8R,10R, 11R,12S,13S,14R)-13-((2,6-dideoxy-3-C-methyl-3-O-methyl-4-C-((propylamino)-methyl)-α-L-ribo-hexopyranosyl)oxy-2-ethyl-3,4,10-trihydroxy-3,5,8,10,12,14-hexamethyl-11-((3,4,6-trideoxy-3-(dimethylamino)-β-D-xylo-hexopyranosyl)oxy)-1-oxa-6-azacyclopentadecan-15-one, and (3R,6R,8R,9R,10S,11S,12R)-1-((2,6-dideoxy-3-C-methyl-3-O-methyl-4-C-((propylamino)methyl-α-L-ribo-hexopyranosyl)oxy)-2-((1R,2R)-1,2-dihydroxy- 1-methylbutyl)-8-hydroxy-3,6,8,10,12-pentamethyl-9-((3,4,6-trideoxy-3-(dimethylamino)-β-D-xylo-hexopyranosyl)oxy)-1-oxa-4-azacyclotridecan-13-one and where the antigenic agents are Pasteurella multocida, Mannheimia haemolytica, Haemophilius somni, and Pasteurella haemolytica.

40. A method of claim 38 where the antimicrobial agent is at least one adjuvant component of a co-administration of an antimicrobial agents and an antigen, where the antimicrobial agent is selected from; penicillin g, procaine, benzathine, penicillin v, cloxacillin, ampicillin sodium, ampicillin, amoxicillin, pivampicillin, carbenicillin, piperacillin, ticarcillin, ureidopenicillin, dzlocillin, temocillin, nafcillin, aminobenzylpenicillius, mecillinam, carboxypenicillin, cephradine, cephalothin, cephapirin, cefazolin, cephalexin, cefaclor, cephadrine, cefadroxil, cefoperazone, cefoxitin, ceftiofur, ceftizoxime, ceftriaxone, cefuroxime, cefquinome, cefotaxime, ceftriaxone, ceftazidime, clavulanate-amoxicillin, clavulanate-ticarcillin, sulbactam-ampicillin, piperacillin-tazobactam, amikacin^{b}, apramycin, gentamicin, kanamycin, neomycin, spectomycin, streptomycin, tobramycin, lincosamides, pleuromutilium, chloramphenicols, macrolides, lincosamides-lincomycine, clindamycin, pirlimycine, pleuromutilins—tiamulin, valnemulin, chloramphenicol, thiaphenicol, florfenical, macrolides—erythromycin, tylasin, spiramycin, tilmicosin, roxithromycin, azithromycin, clarithromycin, ketolide, tulathromycin, oxytetracycline, doxycycline, tetracycline, tetracycline hcl, oxytetracycline hcl, minocycline hcl, doxycycline hyciate, sulfamethazine, trisulfapyrimidine, sulfamethoxazole, sulfadimethoxine, sulfadiazine, sulfisoxazole, phthalylsulfathiazole, salicylazoisulfapyridine, silver sulfadiazine, enrofloxacin, orbifloxacin, difloxacin, danofloxacin, marbofloxacin, sarafloxacin, spectinomycin, imipenem, meropenem, cefotetan, cefprozil, loracarbef, cefdinir, cefpodoxime, ceftibuten, ceftozoxime, cefepime, dirithromycin, dicloxacillin, oxacillin, mezlocillin, nalidixic acid, ciprofloxacin, enoxacin, lomefloxacin, norfloxacin, ofloxacin, levofoxacin, sparfloxacin, alatrofloxacin, gatifloxacin, moxifloxacin, trimethoprim, aztreonam, quinupristin, fosfomycin, metronidazole, nitrofurantoin, rifampin, vancomycin, (2R,3S,4R,5R,8R,10R,11R,12S,13S,14R)-13-((2,6-dideoxy-3-C-methyl-3-O-methyl-4-C-((propylamino)-methyl)-α-L-ribo-hexopyranosyl)oxy-2-ethyl-3,4,10-trihydroxy-3,5,8,10,12,14-hexamethyl-11-((3,4,6-trideoxy-3-(dimethylamino)-β-D-xylo-hexopyranosyl)oxy)-1-oxa-6-azacyclopentadecan-15-one, and (3R,6R,8R,9R,10S,11S,12R)-11-((2,6-dideoxy-3-C-methyl-3-O-methyl-4-C-((propylamino)methyl-α-L-ribo-hexopyranosyl)oxy)-2-((1R,2R)-1,2-dihydroxy-1-methylbutyl)-8-hydroxy-3,6,8,10,12-pentamethyl-9-((3,4,6-trideoxy-3-(dimethylamino)-β-D-xylo-hexopyranosyl)oxy)-1-oxa-4-azacyclotridecan-13-one and where the antigenic agents are Pasteurella multocida, Mannheimia haemolytica, Haemophilus somni, and Pasteurella haemolytica.

41. A method of preventing a disease caused by a pathogenic agent, cancerous cell, or allergen in an animal comprising the step of administering the adjuvant compositions or vaccines described herein and in claim 19 to an animal susceptible to said disease.

42. A method of preventing a disease caused by a pathogenic agent, cancerous cell, or allergen in an animal comprising the step of administering the adjuvant compositions or vaccines described herein and in claim 22 to an animal susceptible to said disease.

43. A method of preventing a disease caused by a pathogenic agent, cancerous cell, or allergen in an animal comprising the step of administering the adjuvant compositions or vaccines described herein and in claim 24 to an animal susceptible to said disease.
